(19) United States
(12) Patent Application Publication
(10) Pub. No.: US 2025/0052747 A1
(43) Pub. Date: Feb. 13, 2025

(54) BIOFLUID-BASED DIAGNOSTIC SCREENING SYSTEM AND METHODS OF USE

(71) Applicant: CellectGen, Inc., Pasadena, CA (US)

(72) Inventors: Robert Gellibolian, Glendale, CA (US); Adam Markaryan, Glendale, CA (US)

(73) Assignee: CellectGen, Inc., Pasadena, CA (US)

(21) Appl. No.: 18/719,809

(22) PCT Filed: Jan. 2, 2023
(86) PCT No.: PCT/US2023/010012

§ 371 Date: Jun. 13, 2024

Related U.S. Application Data
(60) Provisional application No. 63/297,595, filed on Jan. 7, 2022.

(51) Int. Cl. G01N 33/543; G01N 33/573

(52) U.S. Cl. CPC G01N 33/54388; G01N 33/573; G01N 2333/96419

(57) ABSTRACT

A biofluid-based diagnostic screening system and associated methods are disclosed for screening a volume of biofluid of a patient for an at least one medical condition. In at least one embodiment, the system provides a lateral flow test strip, a test housing sized and configured for removably receiving the test strip therewithin, and a user application residing locally in memory on an imaging device and configured for screening a volume of biofluid of a patient, as deposited on the test strip, for an at least one medical condition.

### RELATED APPLICATIONS

This application claims priority and is entitled to the filing date of U.S. provisional application Ser. No. 63/297,595, filed on Jan. 7, 2022. The contents of the aforementioned application are incorporated herein by reference.

### BACKGROUND

The subject of this patent application relates generally to medical diagnostics, and more particularly to a biofluid-based diagnostic screening system and associated methods of use.

Applicant hereby incorporates herein by reference any and all patents and published patent applications cited or referred to in this application.

By way of background, biofluid testing is a diagnostic technique that traditionally involves laboratory analysis of biofluid (such as saliva, urea and blood for example) to identify markers of endocrine, immunologic, inflammatory, infectious, and other types of conditions. Saliva is a useful biofluid for assaying steroid hormones such as cortisol, genetic material like RNA, proteins such as enzymes and antibodies, and a variety of other substances. Saliva testing can be used to screen for or diagnose numerous conditions and disease states, including Cushing's disease, anovulation, HIV, cancer, parasites, hypogonadism, allergies, and periodontal disease. Oftentimes, in order to have any clinical or prognostic value, a biofluid test must be able to measure and track longitudinal trends in data for a given patient—i.e., performing chronobiological assessments that span hours, days, or weeks. For example, to assess success of a given treatment, a medical service provider must determine how biomarker levels might change before and after said treatment, as well as how biomarker levels might change over time. Such assessments require that patients return to the medical service provider for further biofluid testing after the initial treatment, the compliance for which can be difficult to ensure, given that patients are often too busy or unwilling to schedule return visits to their medical service provider.

Accordingly, there remains a need for a consumer-friendly, biofluid-based diagnostic screening system that is configured for being used by a patient without requiring the direct assistance of a medical service provider, thereby allowing the patient to use the device at their convenience and in the comfort of a location of their choosing (i.e., home, office, etc.). Aspects of the present invention fulfill these needs and provide further related advantages as described in the following summary.

It should be noted that the above background description includes information that may be useful in understanding aspects of the present invention. It is not an admission that any of the information provided herein is prior art or relevant to the presently claimed invention, or that any publication specifically or implicitly referenced is prior art.

### SUMMARY

Aspects of the present invention teach certain benefits in construction and use which give rise to the exemplary advantages described below.

The present invention solves the problems described above by providing a biofluid-based diagnostic screening system and associated methods of use for screening a volume of biofluid of a patient for an at least one medical condition. In at least one embodiment, the system provides a lateral flow test strip providing a rigid backing on which is positioned an at least one sample pad configured for receiving and absorbing a volume of the patient's biofluid, a corresponding at least one detection zone containing an at least one biological component for detecting a presence or absence of an at least one target analyte in the volume of the patient's biofluid, the at least one biological component immobilized and arrayed into each of a test line and a control line within said detection zone, and an identification code that identifies a test type to be performed on the volume of the patient's biofluid by the system using the test strip. A test housing is sized and configured for removably receiving the test strip therewithin. The test housing provides a substantially planar top panel configured for temporarily supporting an imaging device thereon, an opposing, substantially planar bottom panel, and a sidewall extending vertically between the top and bottom panels, along a perimeter edge of the top and bottom panels, the top panel, bottom panel and sidewall cooperating to form an enclosed space within the test housing. A strip placeholder is positioned on an upper surface of the bottom panel and configured for assisting in the proper positioning of the test strip within the test housing. An upper surface of the top panel provides an imaging aperture positioned so as to be substantially vertically aligned with the at least one detection zone of the test strip when the test strip is positioned on the strip placeholder within the test housing, the imaging aperture sized for providing an image capture device of the imaging device visual access into the enclosed space within the test housing. An at least one image calibration indicia is positioned on the upper surface of the bottom panel or the backing of the test strip, the image calibration indicia configured for assisting with a calibration of the image capture device of the imaging device. A user application resides locally in memory on the imaging device and is configured for, upon an appropriate volume of the patient's biofluid being deposited onto the at least one sample pad of the test strip, the test strip subsequently being positioned on the strip placeholder within the test housing, and the imaging device being positioned on the upper surface of the top panel of the test housing so that the image capture device is positioned over the imaging aperture of the top panel: capturing an image of the identification code of the test strip using the image capture device of the imaging device; determining the at least one test type to be performed on the volume of the patient's biofluid based on the captured image of the identification code; obtaining a local temperature relative to the imaging device; setting an appropriate calibration curve metric and a timer duration for each of the at least one test type, based on the at least one target analyte of the at least one test type as a function of the local temperature; and for each of the at least one test type to be performed on the volume of the patient's biofluid, upon the associated timer duration being reached for said test type: capturing, via the image capture device of the imaging device, at least one image of the corresponding at least one detection zone of the test strip and the image calibration indicia; adjusting a white balance of the captured at least one image of said detection zone based on the image calibration indicia; calculating a pixel density value of the test line of said detection zone, based on an intensity of said test line depicted in the at least one image; obtaining an analyte concentration value corresponding to the pixel density value of the test line of said detection zone; and communicating the obtained analyte concentration value to at least one of the patient or a medical service provider of the patient.

Other features and advantages of aspects of the present invention will become apparent from the following more detailed description, taken in conjunction with the accompanying drawings, which illustrate, by way of example, the principles of aspects of the invention.

### BRIEF DESCRIPTION OF THE DRAWINGS

The accompanying drawings illustrate aspects of the present invention. In such drawings:

FIG. 1 is a perspective view of an exemplary test housing of an exemplary biofluid-based diagnostic screening system, in accordance with at least one embodiment;

FIG. 2 is a perspective view of an exemplary lateral flow test strip and an exemplary biofluid collection device of an exemplary biofluid-based diagnostic screening system, in accordance with at least one embodiment;

FIG. 3 is a further perspective view of the exemplary biofluid-based diagnostic screening system, in accordance with at least one embodiment; and

FIG. 4 is a flow diagram of an exemplary method for performing a biofluid-based diagnostic screening using the system, in accordance with at least one embodiment.

The above described drawing figures illustrate aspects of the invention in at least one of its exemplary embodiments, which are further defined in detail in the following description. Features, elements, and aspects of the invention that are referenced by the same numerals in different figures represent the same, equivalent, or similar features, elements, or aspects, in accordance with one or more embodiments.

### DETAILED DESCRIPTION

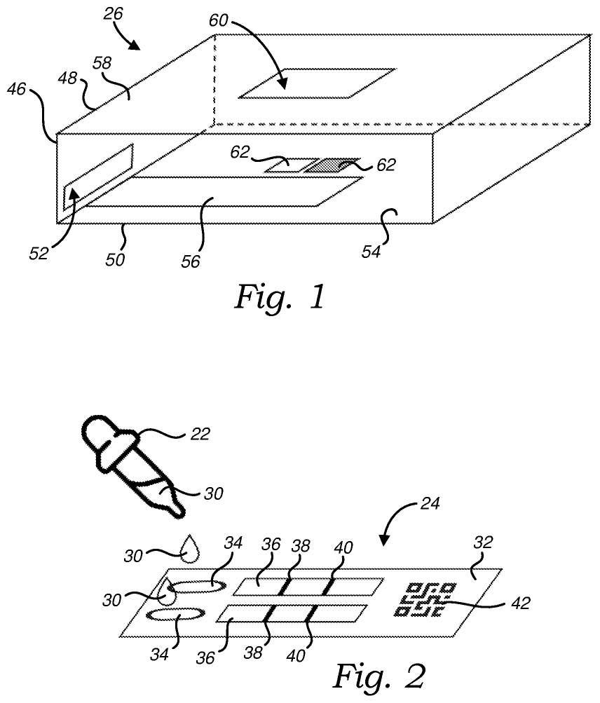

Turning now to FIGS. 1-3, there are shown perspective views of an exemplary embodiment of a biofluid-based diagnostic screening system 20 comprising a biofluid collection device 22, a lateral flow test strip 24, a test housing 26, and an imaging device 28, with the system 20 configured as a consumer-friendly solution for assisting medical service providers and patients with quickly and easily screening for and/or diagnosing medical conditions. In that regard, it should be noted that while the system 20 is shown and described herein in the context of diagnosing periodontal disease for illustrative purposes, the system 20 should not be read as being so limited. In further embodiments, the system 20 may be configured for screening for and/or diagnosing any other medical condition (now known or later discovered) capable of being diagnosed via a patient's biofluid (such as saliva, urea or blood for example) 30. It should also be noted that, in at least one embodiment, the term “medical service provider” is intended to generally include (but is in no way limited to) physicians, dentists, nurses, clinicians, hospitals, clinics, public health departments, and any other type of medical or dental professional or medical/dental entity who may be tasked or otherwise authorized to provide medical services to the at least one patient-even though certain types of medical professionals or entities (such as dentists and dental offices) may be expressly mentioned herein for illustrative purposes.

In at least one embodiment, as illustrated best in FIG. 2, the biofluid collection device 22 is configured for collecting a volume of the patient's biofluid 30 and subsequently depositing an appropriate volume of the biofluid 30 (such as a few drops, for example) onto the test strip 24, as discussed further below. In at least one embodiment, the biofluid collection device 22 is a biofluid collection and filtration device similar to those described in U.S. Pat. Nos. 9,816,087, 10,302,535, and 10,830,677, the details of which are incorporated herein by reference. However, in further embodiments, the biofluid collection device 22 may be any other device (now known or later developed) capable of collecting a volume of biofluid 30 and depositing an appropriate volume of the biofluid 30 onto the test strip 24.

In at least one embodiment, as illustrated best in FIG. 2, the test strip 24 is configured for measuring an amount of matrix metalloproteinase (“MMP”) in the patient's biofluid 30, also known as collagenase-2 or neutrophil collagenase, which is a proteolytic enzyme believed to be responsible for active periodontal soft and hard tissue degeneration. In other words, MMP-8 is an identified biomarker of tissue and bone destruction. Biologically, MMP-8 is generally found in two main forms: a latent (inactive) form of the enzyme (“latent MMP-8”), and an active form of the enzyme (“active MMP-8”). The relationship between these two forms can be described by the following equation:

$$total MMP - 8 = ( active MMP - 8 ) + ( latent MMP - 8 )$$

Thus, the total MMP-8 is comprised of both the latent MMP-8 and the active MMP-8. The latent MMP-8 (75 kDa) contains a pro-domain that masks accessibility of the active site. Studies of anaerobic periodontal infections have shown that active MMP-8 in gingival crevicular biofluid is associated with the degradation of periodontal tissues in progressive periodontitis whereas the latent enzyme is predominant in gingivitis. MMP-8 is produced by various tissues and cells and is secreted by neutrophils. It is synthesized as pre-proMMP-8, from which the signal peptide is removed during translation to generate proMMP-8, which is then secreted in its latent (inactive) form. Once latent MMP-8 is secreted (or degranulated) from neutrophils as a 75 kDa molecule in pro-form (latent MMP-8), it is reduced to its 57 kDa active form (active MMP-8), which is the form believed to be involved in destruction of soft (gums) and hard (bone) tissues and leads to bone loss.

In at least one such embodiment, the test strip 24 is configured for measuring each of the total MMP-8 and the active MMP-8 in the patient's biofluid 30, whereby the ratio of active MMP-8 to total MMP-8 depicts the levels of active disease or disease activity. In at least one further embodiment, the test strip 24 is further configured for measuring levels of a ubiquitous protein (such as actin or alpha-amylase, for example), thereby enabling the system 20 to determine a measure of specific activity of MMP-8 in the biofluid 30—which is typically depicted as units of enzyme per unit of ubiquitous protein in the biofluid 30 (such as milligrams of enzyme per milligram of ubiquitous protein, for example)—and, in turn, normalize the levels of MMP-8 per unit of ubiquitous protein in each sample of biofluid 30, as discussed further below.

With continued reference to FIG. 2, in at least one embodiment, the test strip 24 provides a relatively rigid backing 32 on which is positioned an at least one sample pad 34 configured for receiving and absorbing the appropriate volume of biofluid 30, a corresponding at least one detection zone 36 containing the necessary biological components (such as antigens or antibodies, for example) for detecting the presence or absence of a target analyte (such as MMP-8, for example) in the patient's biofluid 30, immobilized and arrayed in each of a test line 38 and a control line 40 within said detection zone 36, and an identification code 42 that identifies to the imaging device 28 the specific type of test or tests being analyzed (e.g., active MMP-8 test, total MMP-8 test, etc.), the purpose for which is discussed further below. In at least one embodiment, the identification code 42 is a visual code—such as a QR code or barcode, for example—such that the imaging device 28 may obtain the visual code via a camera 44 or other image capture device provided by the imaging device 28. In at least one alternate embodiment, the identification code 42 is an analog or digital signal transmitted from a transponder or transceiver (not shown) provided by the test strip 24—such as an RFID chip, for example—to a transceiver provided by the imaging device 28. In at least one further alternate embodiment, the identification code 42 is an acoustic signal (either audible or inaudible) produced via a speaker (not shown) provided by the test strip 24, such that the imaging device 28 may obtain the acoustic signal via a microphone or other audio capture device provided by the imaging device 28. In still further embodiments, communication of the identification code 42 from the test strip 24 to the imaging device 28 may be achieved using any other techniques, now known or later developed, so long as the system 20 is capable of substantially carrying out the functionality described herein. In at least one alternate embodiment, the identification code 42 is provided by the test housing 26 rather than the test strip 24.

In at least one embodiment, where the test strip 24 is configured for measuring MMP-8 in the biofluid 30, the test line 38 of the at least one detection zone 36 is comprised of the antibodies for active, total and/or latent MMP-8, dependent on the specific type of MMP-8 being measured by said test line 38. In at least one such embodiment, the test line 38 of the at least one detection zone 36 is comprised of a labeled antibody (such as an immunogold antibody, colloidal carbon, colored latex particles, fluorescent labels, etc.), which is amenable for use with the imaging device 28 as it offers a visual signal that is more readily detectable by the imaging device 28, as discussed further below. In at least one alternate such embodiment, the test line 38 of the at least one detection zone 36 is comprised of an enzyme amplified antibody (i.e., an enzyme labeled antibody with a substrate causing signal amplification). In at least one embodiment, the test strip 24 is configured for detecting the presence or absence of more than one target analyte in the patient's biofluid 30—such as both total MMP-8 and active MMP-8, for example—in which case the test strip 24 provides a plurality of sample pads 34 and corresponding detection zones 36. Accordingly, it should be noted that the specific size, shape and dimensions of the test strip 24—along with the specific sizes, shapes, dimensions, quantities and relative positions of the at least one sample pad 34 and corresponding detection zone 36—depicted in the drawings are merely illustrative. In further embodiments, the test strip 24 may take on any other sizes, shapes and/or dimensions, and each of the at least one sample pad 34 and corresponding detection zone 36 may similarly take on any other sizes, shapes, dimensions, quantities and/or relative positions, now known or later developed—dependent at least in part on the particular medical condition to be detected by the system 20—so long as the system 20 is capable of substantially carrying out the functionality described herein. Additionally, in further embodiments, the test strip 24 is configured for detecting the presence or absence of any other target analyte in the patient's biofluid 30 (now known or later discovered), dependent at least in part on the particular medical condition to be detected by the system 20.

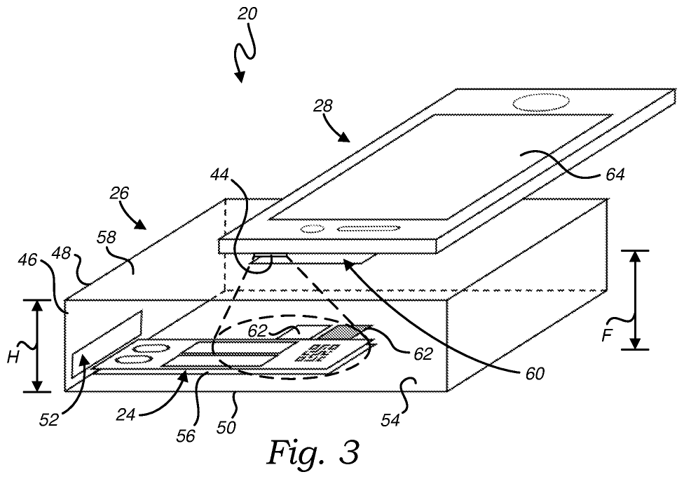

In at least one embodiment, as best illustrated in FIGS. 1 and 3 (in which a portion of a sidewall 46 of the test housing 26 is omitted for illustrative purposes), the test housing 26 is sized and configured for creating an optimal environment in which the imaging device 28 may automatically capture one or more images of the results of the at least one detection zone 36 of the test strip 24 for further automated processing and analysis, as discussed further below. In at least one embodiment, the test housing 26 provides a top panel 48, an opposing bottom panel 50, and a sidewall 46 extending vertically therebetween along a perimeter edge of the opposing top and bottom panels 48 and 50. Accordingly, in at least one embodiment, the top panel 48, bottom panel 50 and sidewall 46 cooperate to form an enclosed space within the test housing 26. Additionally, in at least one embodiment, where the system 20 is provided to the patient or medical service provider as a packaged product, the test housing 26 may be used as a container for temporarily storing one or more of the biofluid collection device 22, test strip 24, and imaging device 28, along with any accompanying documentation. In at least one embodiment, the test housing 26 is constructed out of one or more of paper, cardboard, corrugated cardboard, plastic (such as polyethylene or polypropylene, for example), biodegradable components (such as hemp, for example), or metal. In still further embodiments, the test housing 26 may be constructed out of any other materials (or combinations of materials), now known or later developed, so long as the system 20 is capable of substantially carrying out the functionality described herein. In at least one embodiment, one or more of the top panel 48, bottom panel 50 and sidewall 46 (or select portions thereof) of the test housing 26 are constructed out of a translucent material so as to allow an amount of diffused natural light to enter the enclosed space within the test housing 26 during use of the system 20, the purpose for which is discussed further below. In at least one alternate embodiment, each of the top panel 48, bottom panel 50 and sidewall 46 of the test housing 26 are constructed out of a substantially opaque material.

In at least one embodiment, the sidewall 46 of the test housing 26 provides a strip aperture 52 sized for receiving the test strip 24 therethrough and configured for allowing the test strip 24 to be removably positioned within the test housing 26. In at least one alternate embodiment, the strip aperture 52 is omitted, and the top panel 48 of the test housing 26 is instead configured as a lid capable of being selectively opened and closed. In at least one embodiment, an upper surface 54 of the bottom panel 50 provides a strip placeholder 56 positioned and configured for assisting in the proper positioning of the test strip 24 within the test housing 26. In at least one such embodiment, the strip placeholder 56 is comprised of visual indicia that is printed on or otherwise affixed to the upper surface 54 of the bottom panel 50. In at least one alternate embodiment, the strip placeholder 56 is a physical structure (such as a perimeter wall or ridge, for example) sized for approximating the dimensions of the test strip 24 so as to removably receive the test strip 24 therewithin. In at least one embodiment, the strip placeholder 56 is positioned substantially adjacent to the strip aperture 52.

In at least one embodiment, as illustrated in FIG. 3, an upper surface 58 of the top panel 48 is configured for temporarily supporting the imaging device 28 thereon. Additionally, in at least one embodiment, the top panel 48 provides an imaging aperture 60 positioned so as to be substantially vertically aligned with the at least one detection zone 36 of the test strip 24 when the test strip 24 is positioned on the strip placeholder 56 within the test housing 26. The imaging aperture 60 is sized for providing the camera 44 of the imaging device 28 visual access into the enclosed space within the test housing 26. Accordingly, with the test strip 24 positioned on the strip placeholder 56 within the test housing 26 and the imaging device 28 positioned on the upper surface 58 of the top panel 48 such that the camera 44 of the imaging device 28 is positioned over the imaging aperture 60, the camera 44 of the imaging device 28 is substantially vertically aligned with the at least one detection zone 36 of the test strip 24, the importance of which is discussed further below. In at least one embodiment, the test housing 26 has a height H that is substantially equal to or greater than a focal length F of the camera 44 of the imaging device 28, thereby allowing the camera 44 to more easily and clearly capture one or more images of the test line 38 of the at least one detection zone 36 when the imaging device 28 is positioned on the upper surface 58 of the top panel 48, as also discussed further below.

In at least one embodiment, the upper surface 54 of the bottom panel 50 further provides an at least one image calibration indicia 62 positioned and configured for assisting the camera 44 of the imaging device 28 with calibrating the camera 44 prior to the camera 44 capturing the test line 38 of the at least one detection zone 36 of the test strip 24 during use of the system 20. In at least one embodiment, the image calibration indicia 62 is configured for providing one or more reference colors which allow the imaging device 28 to automatically calibrate a white balance setting and an exposure setting of the camera 44 which, in turn, allows the imaging device 28 to standardize any images that are captured of the test line 38 of the at least one detection zone 36 of the test strip 24 while positioned within the consistent environment created by the enclosed space within the test housing 26, so as to better ensure more accurate and standardized/consistent processing and analysis of the images by the imaging device 28. In at least one embodiment, the image calibration indicia 62 is positioned substantially adjacent to the strip placeholder 56, proximal to the at least one detection zone 36 of the test strip 24 when the test strip 24 is positioned on the strip placeholder 56, thereby allowing the imaging device 28 to automatically calibrate the camera 44 using the image calibration indicia 62 and subsequently capture one or more images of the test line 38 of the at least one detection zone 36 when the imaging device 28 is positioned on the upper surface 58 of the top panel 48, without having to reposition the imaging device 28. Alternatively, as discussed further below, the image calibration indicia 62 may be used to adjust the white balance of the images after they have been captured by the camera 44. In at least one alternate embodiment, the image calibration indicia 62 may be positioned elsewhere on the test housing 26. In at least one further alternate embodiment, the image calibration indicia 62 is positioned on the backing 32 of the test strip 24 rather than on the test housing 26. Similarly, it should be noted that the specific size, shape and dimensions of the test housing 26—along with the specific sizes, shapes, dimensions, quantities and relative positions of the strip aperture 52, strip placeholder 56, imaging aperture 60, and image calibration indicia 62—depicted in the drawings are merely illustrative. In further embodiments, the test housing 26 may take on any other sizes, shapes and/or dimensions, and each of the strip aperture 52, strip placeholder 56, imaging aperture 60, and image calibration indicia 62 may similarly take on any other sizes, shapes, dimensions, quantities and/or relative positions, now known or later developed—dependent at least in part on the particular medical condition to be detected by the system 20—so long as the system 20 is capable of substantially carrying out the functionality described herein. Additionally, in at least one embodiment, where the test housing 26 is used as a container for temporarily storing one or more of the biofluid collection device 22, test strip 24, and imaging device 28, along with any accompanying documentation, one or both of the strip aperture 52 and imaging aperture 60 are formed in the sidewall 46 and top panel 48, respectively, as perforated cutouts, thereby allowing the patient or medical service provider to remove the perforations prior to using the system 20.

In at least one embodiment, as illustrated in FIG. 3, the imaging device 28 is in the possession and/or control of either a medical service provider or a patient of the medical service provider (collectively referred to herein as a “user”) who is desirous of utilizing the system 20 to perform a biofluid-based diagnostic screening using the patient's biofluid 30. It should be noted that the term “imaging device 28” is intended to include any type of computing or electronic device, now known or later developed, capable of carrying out the functionality described herein—such as desktop computers, browser extensions, mobile phones, smartphones, laptop computers, tablet computers, personal data assistants, gaming devices, wearable devices, etc. As such, the system 20 should not be read as being limited to use with any one particular type of computing or electronic device, even though certain exemplary devices may be mentioned or shown herein for illustrative purposes.

In at least one embodiment, the imaging device 28 contains the hardware and software necessary to carry out the exemplary methods for performing a biofluid-based diagnostic screening in conjunction with the other components of the system 20, as described herein. In at least one alternate embodiment, the imaging device 28 is in selective communication with a remote computing system—such as a server, for example—configured for cooperating with the imaging device 28 to carry out the exemplary methods for performing a biofluid-based diagnostic screening described herein. In at least one such alternate embodiment, communication between the imaging device 28 and remote computing system may be achieved using any wired- or wireless-based communication protocol (or combination of protocols) now known or later developed. As such, the present invention should not be read as being limited to any one particular type of communication protocol, even though certain exemplary protocols may be mentioned herein for illustrative purposes, such as the Internet or a local area network for example. Additionally, in at least one such alternate embodiment, the remote computing system comprises a plurality of computing devices selectively working in concert with one another to carry out the exemplary methods for performing a biofluid-based diagnostic screening. In at least one embodiment, the imaging device 28 provides a user application residing locally in memory on the imaging device 28 (either as a standalone application or as a browser extension for an existing Internet browser on the imaging device 28), the user application being configured for carrying out the exemplary methods for performing a biofluid-based diagnostic screening, as discussed further below. It should be noted that the term “memory” is intended to include any type of electronic storage medium (or combination of storage mediums) now known or later developed, such as local hard drives, RAM, flash memory, secure digital (“SD”) cards, external storage devices, network or cloud storage devices, integrated circuits, etc.

In at least one embodiment, the various components of the imaging device 28 may reside on a single computing and/or electronic device, or may separately reside on two or more computing and/or electronic devices in communication with one another. In at least one alternate embodiment, the functionality provided by the user application resides remotely in memory on the remote computing system, with the imaging device 28 capable of accessing said functionality via an online portal hosted by (or at least in communication with) the remote computing system, either in addition to or in lieu of the user application residing locally in memory on the imaging device 28. It should be noted that, for simplicity purposes, the functionality provided by the user application will be described herein as such—even though certain embodiments may provide said functionality through an online portal. It should also be noted that, for simplicity purposes, when discussing functionality and the various methods that may be carried out by the system 20 herein, the terms “imaging device” and “user application” are intended to be interchangeable. With continued reference to FIG. 3, in at least one embodiment, the imaging device 28 provides an at least one display screen 64 for providing an at least one graphical user interface to assist the user in possession of the imaging device 28 to access and utilize the various functions provided by the system 20.

As discussed in detail below, the system 20 may be utilized in a variety of contexts, where it is desirable to analyze the patient's biofluid 30 in order to screen for and/or diagnose one or more medical conditions. It should be noted that the below described applications of the system 20 are merely exemplary and are being provided herein for illustrative purposes. As such, the system 20 and associated methods described herein should not be read as being so limited, but instead can be utilized in any context, now known or later conceived, where there is a need for screening for and/or diagnosing one or more medical conditions using the patient's biofluid 30.

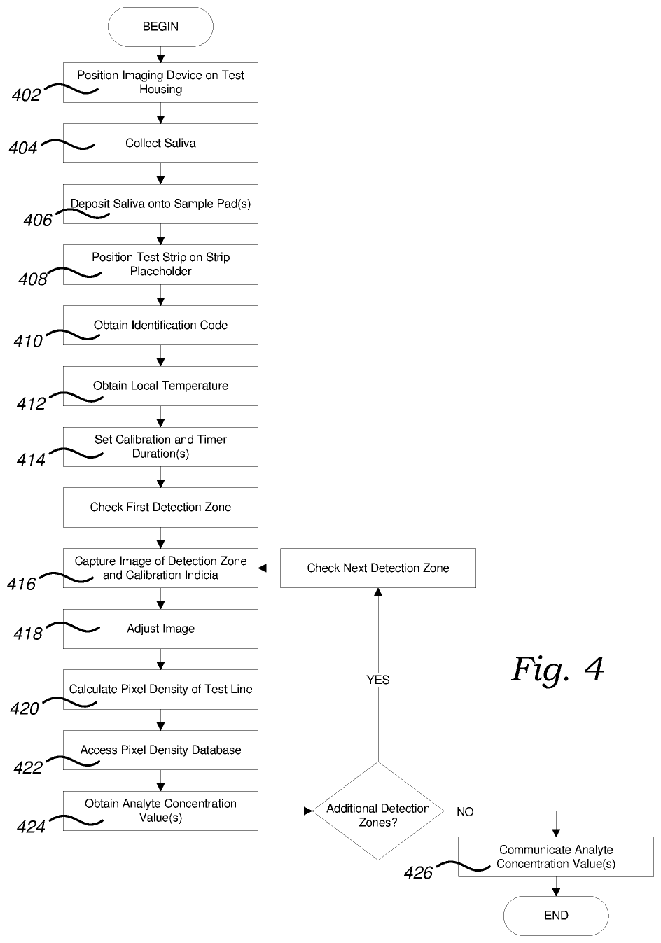

In at least one embodiment, as illustrated in the flow diagram of FIG. 4, upon launching the user application, the user logs in or inputs a unique patient identifier corresponding to the patient along with a unique medical service provider identifier corresponding to the medical service provider, in order to associate the results of the forthcoming biofluid-based test with the patient. The imaging device 28 is then is positioned on the upper surface 58 of the top panel 48 of the test housing 26 so that the camera 44 of the imaging device 28 is positioned over the imaging aperture 60 of the top panel 48 (402). The user then collects a volume of the patient's biofluid 30 (with any debris in the biofluid 30 filtered out) using the biofluid collection device 22 (404) transfers an appropriate volume of the biofluid 30 from the biofluid collection device 22 onto the at least one sample pad 34 of the test strip 24 (406), and the test strip 24 is positioned on the strip placeholder 56 within the test housing 26 (408). In embodiments where the identification code 42 is a visual code positioned on either the test strip 24 or the upper surface 54 of the bottom 20) panel 50 of the test housing 26, the user application automatically determines an at least one test type to be performed by scanning the identification code 42 using the camera 44 of the imaging device 28 (410). In embodiments where the identification code 42 is positioned elsewhere, or is configured as something other than a visual code, the user application determines the at least one test type prior to the imaging device 28 being positioned on the upper surface 58 of the top panel 48 of the test housing 26. The user application also obtains a local temperature relative to the imaging device 28 (412) and automatically sets an appropriate calibration curve metric and a timer duration for each of the at least one test type, based on the target analyte of the at least one test type as a function of the local temperature (414). In at least one embodiment, the calibration curve metric is a mathematical formula (stored locally in memory on the imaging device or remotely) through which the user application is able to derive an analyte concentration value based on a determined pixel density value of the test line 38 of the corresponding detection zone 36 of the test strip 24, as discussed further below. In at least one embodiment, the user application also dynamically generates an appropriate user interface via the display screen 64 of the imaging device 28 based on the at least one test type.

In at least one embodiment, for each of the at least one test type, upon the associated timer duration being reached for said test type, the user application captures, via the camera 44 of the imaging device 28, at least one image of the corresponding detection zone 36 of the test strip 24 and the image calibration indicia 62 (416). The user application then uses the image calibration indicia 62 to adjust a white balance of the captured at least one image (418). In at least one alternate embodiment, the user application uses the image calibration indicia 62 to automatically adjust the white balance setting and exposure setting of the camera 44 prior to the at least one image being captured. With the at least one image properly adjusted based on the image calibration indicia 62, the user application calculates a pixel density value based on an intensity of the test line 38 of the corresponding detection zone 36 of the test strip 24 depicted in the at least one image (420). These steps are repeated for any further test types (and corresponding detection zones 36) that might be present on the test strip 24.

In at least one embodiment, for each of the at least one test type, the user application accesses a database containing pixel density values and corresponding analyte concentration values, and locates a one of the stored pixel density values that most closely matches the pixel density value of the test line 38 of the corresponding detection zone 36 of the test strip 24 depicted in the at least one image (422). In at least one embodiment, the database is stored locally in memory on the imaging device 28. In at least one alternate embodiment, the database may be remotely stored elsewhere, such as in memory on the remote computing system, or on a server in communication with the remote computing system, or on a third-party server, etc. The user application then obtains the analyte concentration value corresponding to the stored pixel density 20) value that most closely matches the pixel density value of the test line 38 of the corresponding detection zone 36 of the test strip 24 depicted in the at least one image (424). In at least one embodiment, upon the user application determining that the pixel density value of the test line 38 of the corresponding detection zone 36 of the test strip 24 depicted in the at least one image closely matches more than one stored pixel density value (i.e., no exact match), the user application obtains the analyte concentration values corresponding to each of said stored pixel density values. In at least one alternate embodiment, upon the user application determining that there are no exact matches between the pixel density value of the test line 38 of the corresponding detection zone 36 of the test strip 24 depicted in the at least one image and the pixel density values stored in the database, the user application obtains the analyte concentration values corresponding to the two stored pixel density values that are immediately higher than and immediately lower than the pixel density value of the test line 38 of the corresponding detection zone 36 of the test strip 24 depicted in the at least one image, with said two obtained analyte concentration values forming an analyte concentration range. In at least one alternate embodiment, for each of the at least one test type, rather than accessing a database containing pixel density values and corresponding analyte concentration values, the user application instead calculates the analyte concentration value for the pixel density value of the test line 38 of the corresponding detection zone 36 of the test strip 24 depicted in the at least one image by using the calibration curve metric as a function of the pixel density value. In at least one such embodiment, the calibration curve metric is a linear equation such as ax+b, where a is slope of the line, x is the analyte concentration value, and b is the intercept). In at least one alternate such embodiment, the calibration curve metric is an S-curve equation. In still further such embodiments, the calibration curve metric could be any other mathematical formula, now known or later developed, capable of correlating pixel density values with their corresponding analyte concentration values.

In at least one embodiment, the user application communicates the obtained analyte concentration value(s) to the patient and/or medical service provider (426). In at least one embodiment, the obtained analyte concentration value(s) is displayed on the display screen 64 of the imaging device 28. In at least one further embodiment, the obtained analyte concentration value(s) is transmitted electronically to the patient and/or medical service provider via email, SMS text, push notification, etc. The medical service provider is then able to further put the obtained analyte concentration value(s) into context with clinical, visual and radiographic assessments and manage patients more effectively in real-time and proactively decide on a treatment plan and visits with the patient. In at least one embodiment, the process is repeated at appropriate time intervals, as often as required or deemed necessary by the medical service provider, thereby providing historical data on analyte levels (separately along with clinical assessment in office) for the patient over time. The historical data will allow medical service providers and patients to visually see whether and when to implement behavior or lifestyle changes to affect risk factors. In at least one embodiment, the user application further generates and provides a graphical representation of the patient's risk factor for the particular medical condition for which the patient is being screened, based on the obtained analyte concentration value(s). Thus, by quantifying analyte concentration values and eliminating the medical service provider's reliance on subjective visual assessments, the system 20 is able to guide treatment in real-time more accurately, more easily, and more cost-effectively than known existing methods. Furthermore, the system 20 requires no special equipment or training on the part of the patient, can be performed anywhere by the patient (which can reduce patient anxiety and increase patient satisfaction and engagement/adherence) and requires no shipping or refrigeration of biofluid 30 samples after collection. can be done by anyone, anywhere.

Aspects of the present specification may also be described as the following embodiments:

1. A biofluid-based diagnostic screening system configured for screening a volume of biofluid of a patient for an at least one medical condition, the system comprising: a lateral flow test strip providing a rigid backing on which is positioned: an at least one sample pad configured for receiving and absorbing a volume of the patient's biofluid; a corresponding at least one detection zone containing an at least one biological component for detecting a presence or absence of an at least one target analyte in the volume of the patient's biofluid, the at least one biological component immobilized and arrayed into each of a test line and a control line within said detection zone; and an identification code that identifies a test type to be performed on the volume of the patient's biofluid by the system using the test strip; a test housing sized and configured for removably receiving the test strip therewithin, the test housing comprising: a substantially planar top panel configured for temporarily supporting an imaging device thereon; an opposing, substantially planar bottom panel; a sidewall extending vertically between the top and bottom panels, along a perimeter edge of the top and bottom panels, the top panel, bottom panel and sidewall cooperating to form an enclosed space within the test housing; a strip placeholder positioned on an upper surface of the bottom panel and configured for assisting in the proper positioning of the test strip within the test housing; an upper surface of the top panel providing an imaging aperture positioned so as to be substantially vertically aligned with the at least one detection zone of the test strip when the test strip is positioned on the strip placeholder within the test housing, the imaging aperture sized for providing an image capture device of the imaging device visual access into the enclosed space within the test housing; and an at least one image calibration indicia positioned on the upper surface of the bottom panel or the backing of the test strip, the image calibration indicia configured for assisting with a calibration of the image capture device of the imaging device; and a user application residing locally in memory on the imaging device, the user application configured for, upon an appropriate volume of the patient's biofluid being deposited onto the at least one sample pad of the test strip, the test strip subsequently being positioned on the strip placeholder within the test housing, and the imaging device being positioned on the upper surface of the top panel of the test housing so that the image capture device is positioned over the imaging aperture of the top panel: capturing an image of the identification code of the test strip using the image capture device of the imaging device; determining the at least one test type to be performed on the volume of the patient's biofluid based on the captured image of the identification code; obtaining a local temperature relative to the imaging device; setting an appropriate calibration curve metric and a timer duration for each of the at least one test type, based on the at least one target analyte of the at least one test type as a function of the local temperature; and for each of the at least one test type to be performed on the volume of the patient's biofluid, upon the associated timer duration being reached for said test type: capturing, via the image capture device of the imaging device, at least one image of the corresponding at least one detection zone of the test strip and the image calibration indicia; adjusting a white balance of the captured at least one image of said detection zone based on the image calibration indicia; calculating a pixel density value of the test line of said detection zone, based on an intensity of said test line depicted in the at least one image; obtaining an analyte concentration value corresponding to the pixel density value of the test line of said detection zone; and communicating the obtained analyte concentration value to at least one of the patient or a medical service provider of the patient.

2. The biofluid-based diagnostic screening system according to embodiment 1, wherein the at least one target analyte is matrix metalloproteinase (“MMP”).

3. The biofluid-based diagnostic screening system according to embodiments 1-2, wherein the at least one biological component of the at least one detection zone comprises antibodies for at least one of active MMP-8, total MMP-8, and latent MMP-8.

4. The biofluid-based diagnostic screening system according to embodiments 1-3, wherein the least one detection zone of the test strip is further configured for measuring a quantity of an at least one ubiquitous protein, thereby enabling the user application to determine a measure of specific activity of MMP-8 in the patient's biofluid and, in turn, normalize the levels of MMP-8 per unit of ubiquitous protein in the patient's biofluid.

5. The biofluid-based diagnostic screening system according to embodiments 1-4, wherein the at least one biological component of the at least one detection zone further comprises a labeled antibody.

6. The biofluid-based diagnostic screening system according to embodiments 1-5, wherein the at least one biological component of the at least one detection zone further comprises an enzyme amplified antibody.

7. The biofluid-based diagnostic screening system according to embodiments 1-6, wherein the test housing is further configured as a container for temporarily storing one or more of the biofluid collection device, test strip, and imaging device.

8. The biofluid-based diagnostic screening system according to embodiments 1-7, wherein one or both of the strip aperture and imaging aperture are formed in the sidewall and top panel, respectively, of the test housing as perforated cutouts.

9. The biofluid-based diagnostic screening system according to embodiments 1-8, wherein one or more of the top panel, bottom panel and sidewall, or select portions thereof, of the test housing are constructed out of a translucent material so as to allow an amount of diffused light from an external environment to enter the enclosed space within the test housing during use of the system.

10. The biofluid-based diagnostic screening system according to embodiments 1-9, wherein the sidewall of the test housing provides a strip aperture sized for receiving the test strip therethrough and configured for allowing the test strip to be removably positioned within the test housing.

11. The biofluid-based diagnostic screening system according to embodiments 1-10, wherein the strip placeholder is positioned substantially adjacent to the strip aperture.

12. The biofluid-based diagnostic screening system according to embodiments 1-11, wherein the top panel of the test housing is configured for being selectively opened and closed.

13. The biofluid-based diagnostic screening system according to embodiments 1-12, wherein the strip placeholder is comprised of visual indicia that is printed on or otherwise affixed to the upper surface of the bottom panel.

14. The biofluid-based diagnostic screening system according to embodiments 1-13, wherein the strip placeholder is a physical structure sized for approximating the dimensions of the test strip so as to removably receive the test strip therewithin.

15. The biofluid-based diagnostic screening system according to embodiments 1-14, wherein the test housing has a height that is substantially equal to or greater than a focal length of the image capture device of the imaging device.

16. The biofluid-based diagnostic screening system according to embodiments 1-15, wherein the at least one image calibration indicia provides an at least one reference color.

17. The biofluid-based diagnostic screening system according to embodiments 1-16, wherein the at least one image calibration indicia is positioned substantially adjacent to the strip placeholder, proximal to the at least one detection zone of the test strip when the test strip is positioned on the strip placeholder.

18. The biofluid-based diagnostic screening system according to embodiments 1-17, wherein the step of the user application obtaining an analyte concentration value corresponding to the pixel density value of the test line of said detection zone, further comprises the steps of the user application: accessing a database containing a plurality of pixel density values and corresponding analyte concentration values; locating at least one of the stored pixel density values in the database that most closely matches the pixel density value of the test line of said detection zone; and obtaining the analyte concentration value corresponding to each of the at least one of the stored pixel density values in the database that most closely matches the pixel density value of the test line of said detection zone.

19. The biofluid-based diagnostic screening system according to embodiments 1-18, wherein the step of the user application obtaining the analyte concentration value corresponding to each of the at least one of the stored pixel density values in the database that most closely matches the pixel density value of the test line of said detection zone, further comprises the steps of the user application: obtaining the analyte concentration value corresponding to a one of the stored pixel density values in the database that is immediately higher than the pixel density value of the test line of said detection zone; obtaining the analyte concentration value corresponding to a one of the stored pixel density values in the database that is immediately lower than the pixel density value of the test line of said detection zone; and calculating an analyte concentration range for the test line of said detection zone based on the analyte concentration values corresponding to the immediately higher pixel density value and the immediately lower pixel density value in the database.

20. The biofluid-based diagnostic screening system according to embodiments 1-19, wherein the step of the user application obtaining an analyte concentration value corresponding to the pixel density value of the test line of said detection zone, further comprises the step of the user application calculating the analyte concentration value by using the calibration curve metric as a function of the pixel density value.

21. The biofluid-based diagnostic screening system according to embodiments 1-20, further comprising a biofluid collection device configured for collecting a volume of the patient's biofluid and subsequently depositing an appropriate volume of the biofluid onto the at least one sample pad of the test strip.

22. The biofluid-based diagnostic screening system according to embodiments 1-21, wherein the biofluid collection device is further configured for filtering out any debris in the appropriate volume of the biofluid prior to depositing said biofluid onto the at least one sample pad of the test strip.

23. The biofluid-based diagnostic screening system according to embodiments 1-22, wherein the identification code is a visual code configured for being obtained by the imaging device via the image capture device of the imaging device.

24. The biofluid-based diagnostic screening system according to embodiments 1-23, wherein the image capture device is a camera.

25. The biofluid-based diagnostic screening system according to embodiments 1-24, wherein the identification code is an analog or digital signal configured for being transmitted from the test strip to a transceiver provided by the imaging device.

26. The biofluid-based diagnostic screening system according to embodiments 1-25, wherein the identification code is an acoustic signal produced via a speaker provided by the test strip and configured for being obtained by an audio capture device provided by the imaging device.

27. A biofluid-based diagnostic screening system configured for screening a volume of biofluid of a patient for an at least one medical condition, the system comprising: a lateral flow test strip providing a rigid backing on which is positioned: an at least one sample pad configured for receiving and absorbing a volume of the patient's biofluid; a corresponding at least one detection zone containing an at least one biological component for detecting a presence or absence of an at least one target analyte in the volume of the patient's biofluid, the at least one biological component immobilized and arrayed into each of a test line and a control line within said detection zone; and an identification code that identifies a test type to be performed on the volume of the patient's biofluid by the system using the test strip; a biofluid collection device configured for collecting a volume of the patient's biofluid and subsequently depositing an appropriate volume of the biofluid onto the at least one sample pad of the test strip; a test housing sized and configured for removably receiving the test strip therewithin, the test housing comprising: a substantially planar top panel configured for temporarily supporting an imaging device thereon; an opposing, substantially planar bottom panel; a sidewall extending vertically between the top and bottom panels, along a perimeter edge of the top and bottom panels, the top panel, bottom panel and sidewall cooperating to form an enclosed space within the test housing; a strip placeholder positioned on an upper surface of the bottom panel and configured for assisting in the proper positioning of the test strip within the test housing; an upper surface of the top panel providing an imaging aperture positioned so as to be substantially vertically aligned with the at least one detection zone of the test strip when the test strip is positioned on the strip placeholder within the test housing, the imaging aperture sized for providing an image capture device of the imaging device visual access into the enclosed space within the test housing; and an at least one image calibration indicia positioned on the upper surface of the bottom panel or the backing of the test strip, the image calibration indicia configured for assisting with a calibration of the image capture device of the imaging device; and a user application residing locally in memory on the imaging device, the user application configured for, upon an appropriate volume of the patient's biofluid being deposited onto the at least one sample pad of the test strip, the test strip subsequently being positioned on the strip placeholder within the test housing, and the imaging device being positioned on the upper surface of the top panel of the test housing so that the image capture device is positioned over the imaging aperture of the top panel: capturing an image of the identification code of the test strip using the image capture device of the imaging device; determining the at least one test type to be performed on the volume of the patient's biofluid based on the captured image of the identification code; obtaining a local temperature relative to the imaging device; setting an appropriate calibration curve metric and a timer duration for each of the at least one test type, based on the at least one target analyte of the at least one test type as a function of the local temperature; and for each of the at least one test type to be performed on the volume of the patient's biofluid, upon the associated timer duration being reached for said test type: capturing, via the image capture device of the imaging device, at least one image of the corresponding at least one detection zone of the test strip and the image calibration indicia; adjusting a white balance of the captured at least one image of said detection zone based on the image calibration indicia; calculating a pixel density value of the test line of said detection zone, based on an intensity of said test line depicted in the at least one image; obtaining an analyte concentration value corresponding to the pixel density value of the test line of said detection zone; and communicating the obtained analyte concentration value to at least one of the patient or a medical service provider of the patient.

28. A method for screening a volume of biofluid of a patient for an at least one medical condition using the biofluid-based diagnostic screening system according to embodiments 1-25, the method comprising the steps of: implementing a user application residing in memory on an imaging device; collecting a volume of the patient's biofluid using the biofluid collection device; depositing an appropriate volume of the biofluid from the biofluid collection device onto the at least one sample pad of the test strip; positioning the test strip on the strip placeholder within the test housing; positioning the imaging device on the upper surface of the top panel of the test housing so that the image capture device is positioned over the imaging aperture of the top panel; the user application capturing an image of the identification code of the test strip using the image capture device of the imaging device; the user application determining the at least one test type to be performed on the volume of the patient's biofluid based on the captured image of the identification code; the user application obtaining a local temperature relative to the imaging device; the user application setting an appropriate calibration curve metric and a timer duration for each of the at least one test type, based on the at least one target analyte of the at least one test type as a function of the local temperature; and for each of the at least one test type to be performed on the volume of the patient's biofluid, upon the associated timer duration being reached for said test type: the user application capturing, via the image capture device of the imaging device, at least one image of the corresponding at least one detection zone of the test strip and the image calibration indicia; the user application adjusting a white balance of the captured at least one image of said detection zone based on the image calibration indicia; the user application calculating a pixel density value of the test line of said detection zone, based on an intensity of said test line depicted in the at least one image; the user application obtaining an analyte concentration value corresponding to the pixel density value of the test line of said detection zone; and the user application communicating the obtained analyte concentration value to at least one of the patient or a medical service provider of the patient.

29. The method according to embodiment 28, wherein the step of positioning the test strip on the strip placeholder within the test housing further comprises the step of inserting the test strip through a strip aperture provided by the sidewall of the test housing.

30. The method according to embodiments 28-29, wherein the step of the user application obtaining an analyte concentration value corresponding to the pixel density value of the test line of said detection zone, further comprises the steps of: the user application accessing a database containing a plurality of pixel density values and corresponding analyte concentration values; the user application locating at least one of the stored pixel density values in the database that most closely matches the pixel density value of the test line of said detection zone; and the user application obtaining the analyte concentration value corresponding to each of the at least one of the stored pixel density values in the database that most closely matches the pixel density value of the test line of said detection zone.

31. The method according to embodiments 28-30, wherein the step of the user application obtaining the analyte concentration value corresponding to each of the at least one of the stored pixel density values in the database that most closely matches the pixel density value of the test line of said detection zone, further comprises the steps of: the user application obtaining the analyte concentration value corresponding to a one of the stored pixel density values in the database that is immediately higher than the pixel density value of the test line of said detection zone; the user application obtaining the analyte concentration value corresponding to a one of the stored pixel density values in the database that is immediately lower than the pixel density value of the test line of said detection zone; and the user application calculating an analyte concentration range for the test line of said detection zone based on the analyte concentration values corresponding to the immediately higher pixel density value and the immediately lower pixel density value in the database.

32. The method according to embodiments 28-31, wherein the step of the user application obtaining an analyte concentration value corresponding to the pixel density value of the test line of said detection zone, further comprises the step of the user application calculating the analyte concentration value by using the calibration curve metric as a function of the pixel density value.

33. The method according to embodiments 28-32, further comprising the step of filtering out any debris in the volume of the biofluid prior to depositing said biofluid onto the at least one sample pad of the test strip.

In closing, regarding the exemplary embodiments of the present invention as shown and described herein, it will be appreciated that a biofluid-based diagnostic screening system and associated methods of use are disclosed and configured for being used by a patient without requiring the direct assistance of a medical service provider. Because the principles of the invention may be practiced in a number of configurations beyond those shown and described, it is to be understood that the invention is not in any way limited by the exemplary embodiments, but is generally directed to a biofluid-based diagnostic screening system and is able to take numerous forms to do so without departing from the spirit and scope of the invention. It will also be appreciated by those skilled in the art that the present invention is not limited to the particular geometries and materials of construction disclosed, but may instead entail other functionally comparable structures or materials, now known or later developed, without departing from the spirit and scope of the invention.

Certain embodiments of the present invention are described herein, including the best mode known to the inventor(s) for carrying out the invention. Of course, variations on these described embodiments will become apparent to those of ordinary skill in the art upon reading the foregoing description. The inventor(s) expect skilled artisans to employ such variations as appropriate, and the inventor(s) intend for the present invention to be practiced otherwise than specifically described herein. Accordingly, this invention includes all modifications and equivalents of the subject matter recited in the claims appended hereto as permitted by applicable law. Moreover, any combination of the above-described embodiments in all possible variations thereof is encompassed by the invention unless otherwise indicated herein or otherwise clearly contradicted by context.

Groupings of alternative embodiments, elements, or steps of the present invention are not to be construed as limitations. Each group member may be referred to and claimed individually or in any combination with other group members disclosed herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Unless otherwise indicated, all numbers expressing a characteristic, item, quantity, parameter, property, term, and so forth used in the present specification and claims are to be understood as being modified in all instances by the terms “about” and “approximately.” As used herein, the terms “about” and “approximately” mean that the characteristic, item, quantity, parameter, property, or term so qualified encompasses a range of plus or minus ten percent above and below the value of the stated characteristic, item, quantity, parameter, property, or term. Accordingly, unless indicated to the contrary, the numerical parameters set forth in the specification and attached claims are approximations that may vary. At the very least, and not as an attempt to limit the application of the doctrine of equivalents to the scope of the claims, each numerical indication should at least be construed in light of the number of reported significant digits and by applying ordinary rounding techniques. Notwithstanding that the numerical ranges and values setting forth the broad scope of the invention are approximations, the numerical ranges and values set forth in the specific examples are reported as precisely as possible. Any numerical range or value, however, inherently contains certain errors necessarily resulting from the standard deviation found in their respective testing measurements. Recitation of numerical ranges of values herein is merely intended to serve as a shorthand method of referring individually to each separate numerical value falling within the range. Unless otherwise indicated herein, each individual value of a numerical range is incorporated into the present specification as if it were individually recited herein. Similarly, as used herein, unless indicated to the contrary, the term “substantially” is a term of degree intended to indicate an approximation of the characteristic, item, quantity, parameter, property, or term so qualified, encompassing a range that can be understood and construed by those of ordinary skill in the art.

Use of the terms “may” or “can” in reference to an embodiment or aspect of an embodiment also carries with it the alternative meaning of “may not” or “cannot.” As such, if the present specification discloses that an embodiment or an aspect of an embodiment may be or can be included as part of the inventive subject matter, then the negative limitation or exclusionary proviso is also explicitly meant, meaning that an embodiment or an aspect of an embodiment may not be or cannot be included as part of the inventive subject matter. In a similar manner, use of the term “optionally” in reference to an embodiment or aspect of an embodiment means that such embodiment or aspect of the embodiment may be included as part of the inventive subject matter or may not be included as part of the inventive subject matter. Whether such a negative limitation or exclusionary proviso applies will be based on whether the negative limitation or exclusionary proviso is recited in the claimed subject matter.

The terms “a,” “an,” “the” and similar references used in the context of describing the present invention (especially in the context of the following claims) are to be construed to cover both the singular and the plural, unless otherwise indicated herein or clearly contradicted by context. Further, ordinal indicators—such as “first,” “second,” “third,” etc.—for identified elements are used to distinguish between the elements, and do not indicate or imply a required or limited number of such elements, and do not indicate a particular position or order of such elements unless otherwise specifically stated. All methods described herein can be performed in any suitable order unless otherwise indicated herein or otherwise clearly contradicted by context. The use of any and all examples, or exemplary language (e.g., “such as”) provided herein is intended merely to better illuminate the present invention and does not pose a limitation on the scope of the invention otherwise claimed. No language in the present specification should be construed as indicating any non-claimed element essential to the practice of the invention.

When used in the claims, whether as filed or added per amendment, the open-ended transitional term “comprising” (along with equivalent open-ended transitional phrases thereof such as “including,” “containing” and “having”) encompasses all the expressly recited elements, limitations, steps and/or features alone or in combination with un-recited subject matter; the named elements, limitations and/or features are essential, but other unnamed elements, limitations and/or features may be added and still form a construct within the scope of the claim. Specific embodiments disclosed herein may be further limited in the claims using the closed-ended transitional phrases “consisting of” or “consisting essentially of” in lieu of or as an amendment for “comprising.” When used in the claims, whether as filed or added per amendment, the closed-ended transitional phrase “consisting of” excludes any element, limitation, step, or feature not expressly recited in the claims. The closed-ended transitional phrase “consisting essentially of” limits the scope of a claim to the expressly recited elements, limitations, steps and/or features and any other elements, limitations, steps and/or features that do not materially affect the basic and novel characteristic(s) of the claimed subject matter. Thus, the meaning of the open-ended transitional phrase “comprising” is being defined as encompassing all the specifically recited elements, limitations, steps and/or features as well as any optional, additional unspecified ones. The meaning of the closed-ended transitional phrase “consisting of” is being defined as only including those elements, limitations, steps and/or features specifically recited in the claim, whereas the meaning of the closed-ended transitional phrase “consisting essentially of” is being defined as only including those elements, limitations, steps and/or features specifically recited in the claim and those elements, limitations, steps and/or features that do not materially affect the basic and novel characteristic(s) of the claimed subject matter. Therefore, the open-ended transitional phrase “comprising” (along with equivalent open-ended transitional phrases thereof) includes within its meaning, as a limiting case, claimed subject matter specified by the closed-ended transitional phrases “consisting of” or “consisting essentially of.” As such, embodiments described herein or so claimed with the phrase “comprising” are expressly or inherently unambiguously described, enabled and supported herein for the phrases “consisting essentially of” and “consisting of.”

Any claims intended to be treated under 35 U.S.C. § 112(f) will begin with the words “means for,” but use of the term “for” in any other context is not intended to invoke treatment under 35 U.S.C. § 112(f). Accordingly, Applicant reserves the right to pursue additional claims after filing this application, in either this application or in a continuing application.

It should be understood that the logic code, programs, modules, processes, methods, and the order in which the respective elements of each method are performed are purely exemplary. Depending on the implementation, they may be performed in any order or in parallel, unless indicated otherwise in the present disclosure. Further, the logic code is not related, or limited to any particular programming language, and may comprise one or more modules that execute on one or more processors in a distributed, non-distributed, or multiprocessing environment. Additionally, the various illustrative logical blocks, modules, methods, and algorithm processes and sequences described in connection with the embodiments disclosed herein can be implemented as electronic hardware, computer software, or combinations of both. To clearly illustrate this interchangeability of hardware and software, various illustrative components, blocks, modules, and process actions have been described above generally in terms of their functionality. Whether such functionality is implemented as hardware or software depends upon the particular application and design constraints imposed on the overall system. The described functionality can be implemented in varying ways for each particular application, but such implementation decisions should not be interpreted as causing a departure from the scope of this document.

The phrase “non-transitory,” in addition to having its ordinary meaning, as used in this document means “enduring or long-lived”. The phrase “non-transitory computer readable medium,” in addition to having its ordinary meaning, includes any and all computer readable mediums, with the sole exception of a transitory, propagating signal. This includes, by way of example and not limitation, non-transitory computer-readable mediums such as register memory, processor cache and random-access memory (“RAM”).

The methods as described above may be used in the fabrication of integrated circuit chips. The resulting integrated circuit chips can be distributed by the fabricator in raw wafer form (that is, as a single wafer that has multiple unpackaged chips), as a bare die, or in a packaged form. In the latter case, the chip is mounted in a single chip package (such as a plastic carrier, with leads that are affixed to a motherboard or other higher level carrier) or in a multi-chip package (such as a ceramic carrier that has either or both surface interconnections or buried interconnections). In any case, the chip is then integrated with other chips, discrete circuit elements, and/or other signal processing devices as part of either (a) an intermediate product, such as a motherboard, or (b) an end product. The end product can be any product that includes integrated circuit chips, ranging from toys and other low-end applications to advanced computer products having a display, a keyboard or other input device, and a central processor.

All patents, patent publications, and other publications referenced and identified in the present specification are individually and expressly incorporated herein by reference in their entirety for the purpose of describing and disclosing, for example, the compositions and methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicant and does not constitute any admission as to the correctness of the dates or contents of these documents.

While aspects of the invention have been described with reference to at least one exemplary embodiment, it is to be clearly understood by those skilled in the art that the invention is not limited thereto. Rather, the scope of the invention is to be interpreted only in conjunction with the appended claims and it is made clear, here, that the inventor(s) believe that the claimed subject matter is the invention.

What is claimed is:

1. A biofluid-based diagnostic screening system configured for screening a volume of biofluid of a patient for an at least one medical condition, the system comprising:
a lateral flow test strip providing a rigid backing on which is positioned:
an at least one sample pad configured for receiving and absorbing a volume of the patient's biofluid;
a corresponding at least one detection zone containing an at least one biological component for detecting a presence or absence of an at least one target analyte in the volume of the patient's biofluid, the at least one biological component immobilized and arrayed into each of a test line and a control line within said detection zone; and
an identification code that identifies a test type to be performed on the volume of the patient's biofluid by the system using the test strip;
a test housing sized and configured for removably receiving the test strip therewithin, the test housing comprising:
a substantially planar top panel configured for temporarily supporting an imaging device thereon;
an opposing, substantially planar bottom panel;
a sidewall extending vertically between the top and bottom panels, along a perimeter edge of the top and bottom panels, the top panel, bottom panel and sidewall cooperating to form an enclosed space within the test housing;
a strip placeholder positioned on an upper surface of the bottom panel and configured for assisting in the proper positioning of the test strip within the test housing;
an upper surface of the top panel providing an imaging aperture positioned so as to be substantially vertically aligned with the at least one detection zone of the test strip when the test strip is positioned on the strip placeholder within the test housing, the imaging aperture sized for providing an image capture device of the imaging device visual access into the enclosed space within the test housing; and
an at least one image calibration indicia positioned on the upper surface of the bottom panel or the backing of the test strip, the image calibration indicia configured for assisting with a calibration of the image capture device of the imaging device; and
a user application residing locally in memory on the imaging device, the user application configured for, upon an appropriate volume of the patient's biofluid being deposited onto the at least one sample pad of the test strip, the test strip subsequently being positioned on the strip placeholder within the test housing, and the imaging device being positioned on the upper surface of the top panel of the test housing so that the image capture device is positioned over the imaging aperture of the top panel:
capturing an image of the identification code of the test strip using the image capture device of the imaging device;
determining the at least one test type to be performed on the volume of the patient's biofluid based on the captured image of the identification code;
obtaining a local temperature relative to the imaging device;
setting an appropriate calibration curve metric and a timer duration for each of the at least one test type, based on the at least one target analyte of the at least one test type as a function of the local temperature; and
for each of the at least one test type to be performed on the volume of the patient's biofluid, upon the associated timer duration being reached for said test type:
capturing, via the image capture device of the imaging device, at least one image of the corresponding at least one detection zone of the test strip and the image calibration indicia;
adjusting a white balance of the captured at least one image of said detection zone based on the image calibration indicia;
calculating a pixel density value of the test line of said detection zone, based on an intensity of said test line depicted in the at least one image;
obtaining an analyte concentration value corresponding to the pixel density value of the test line of said detection zone; and
communicating the obtained analyte concentration value to at least one of the patient or a medical service provider of the patient.

2. The biofluid-based diagnostic screening system of claim 1, wherein the at least one target analyte is matrix metalloproteinase (“MMP”).

3. The biofluid-based diagnostic screening system of claim 2, wherein the at least one biological component of the at least one detection zone comprises antibodies for at least one of active MMP-8, total MMP-8, and latent MMP-8.

4. The biofluid-based diagnostic screening system of claim 2, wherein the least one detection zone of the test strip is further configured for measuring a quantity of an at least one ubiquitous protein, thereby enabling the user application to determine a measure of specific activity of MMP-8 in the patient's biofluid and, in turn, normalize the levels of MMP-8 per unit of ubiquitous protein in the patient's biofluid.

5. The biofluid-based diagnostic screening system of claim 1, wherein the at least one biological component of the at least one detection zone further comprises a labeled antibody.

6. The biofluid-based diagnostic screening system of claim 1, wherein the at least one biological component of the at least one detection zone further comprises an enzyme amplified antibody.

7. The biofluid-based diagnostic screening system of claim 1, wherein one or more of the top panel, bottom panel and sidewall, or select portions thereof, of the test housing are constructed out of a translucent material so as to allow an amount of diffused light from an external environment to enter the enclosed space within the test housing during use of the system.

8. The biofluid-based diagnostic screening system of claim 1, wherein the sidewall of the test housing provides a strip aperture sized for receiving the test strip therethrough and configured for allowing the test strip to be removably positioned within the test housing.

9. The biofluid-based diagnostic screening system of claim 8, wherein the strip placeholder is positioned substantially adjacent to the strip aperture.

10. The biofluid-based diagnostic screening system of claim 1, wherein the strip placeholder is comprised of visual indicia that is printed on or otherwise affixed to the upper surface of the bottom panel.

11. The biofluid-based diagnostic screening system of claim 1, wherein the strip placeholder is a physical structure sized for approximating the dimensions of the test strip so as to removably receive the test strip therewithin.

12. The biofluid-based diagnostic screening system of claim 1, wherein the test housing has a height that is substantially equal to or greater than a focal length of the image capture device of the imaging device.

13. The biofluid-based diagnostic screening system of claim 1, wherein the at least one image calibration indicia is positioned substantially adjacent to the strip placeholder, proximal to the at least one detection zone of the test strip when the test strip is positioned on the strip placeholder.

14. The biofluid-based diagnostic screening system of claim 1, wherein the step of the user application obtaining an analyte concentration value corresponding to the pixel density value of the test line of said detection zone, further comprises the steps of the user application:
accessing a database containing a plurality of pixel density values and corresponding analyte concentration values;
locating at least one of the stored pixel density values in the database that most closely matches the pixel density value of the test line of said detection zone; and
obtaining the analyte concentration value corresponding to each of the at least one of the stored pixel density values in the database that most closely matches the pixel density value of the test line of said detection zone.

15. The biofluid-based diagnostic screening system of claim 1, wherein the step of the user application obtaining an analyte concentration value corresponding to the pixel density value of the test line of said detection zone, further comprises the step of the user application calculating the analyte concentration value by using the calibration curve metric as a function of the pixel density value.

16. The biofluid-based diagnostic screening system of claim 1, further comprising a biofluid collection device configured for collecting a volume of the patient's biofluid and subsequently depositing an appropriate volume of the biofluid onto the at least one sample pad of the test strip.

17. The biofluid-based diagnostic screening system of claim 1, wherein the identification code is a visual code configured for being obtained by the imaging device via the image capture device of the imaging device.

18. A biofluid-based diagnostic screening system configured for screening a volume of biofluid of a patient for an at least one medical condition, the system comprising:
a lateral flow test strip providing a rigid backing on which is positioned:
an at least one sample pad configured for receiving and absorbing a volume of the patient's biofluid;
a corresponding at least one detection zone containing an at least one biological component for detecting a presence or absence of an at least one target analyte in the volume of the patient's biofluid, the at least one biological component immobilized and arrayed into each of a test line and a control line within said detection zone; and
an identification code that identifies a test type to be performed on the volume of the patient's biofluid by the system using the test strip;
a biofluid collection device configured for collecting a volume of the patient's biofluid and subsequently depositing an appropriate volume of the biofluid onto the at least one sample pad of the test strip;
a test housing sized and configured for removably receiving the test strip therewithin, the test housing comprising:
a substantially planar top panel configured for temporarily supporting an imaging device thereon;
an opposing, substantially planar bottom panel;
a sidewall extending vertically between the top and bottom panels, along a perimeter edge of the top and bottom panels, the top panel, bottom panel and sidewall cooperating to form an enclosed space within the test housing;
a strip placeholder positioned on an upper surface of the bottom panel and configured for assisting in the proper positioning of the test strip within the test housing;
an upper surface of the top panel providing an imaging aperture positioned so as to be substantially vertically aligned with the at least one detection zone of the test strip when the test strip is positioned on the strip placeholder within the test housing, the imaging aperture sized for providing an image capture device of the imaging device visual access into the enclosed space within the test housing; and
an at least one image calibration indicia positioned on the upper surface of the bottom panel or the backing of the test strip, the image calibration indicia configured for assisting with a calibration of the image capture device of the imaging device; and
a user application residing locally in memory on the imaging device, the user application configured for, upon an appropriate volume of the patient's biofluid being deposited onto the at least one sample pad of the test strip, the test strip subsequently being positioned on the strip placeholder within the test housing, and the imaging device being positioned on the upper surface of the top panel of the test housing so that the image capture device is positioned over the imaging aperture of the top panel:
capturing an image of the identification code of the test strip using the image capture device of the imaging device;
determining the at least one test type to be performed on the volume of the patient's biofluid based on the captured image of the identification code;
obtaining a local temperature relative to the imaging device;
setting an appropriate calibration curve metric and a timer duration for each of the at least one test type, based on the at least one target analyte of the at least one test type as a function of the local temperature; and
for each of the at least one test type to be performed on the volume of the patient's biofluid, upon the associated timer duration being reached for said test type:
capturing, via the image capture device of the imaging device, at least one image of the corresponding at least one detection zone of the test strip and the image calibration indicia;
adjusting a white balance of the captured at least one image of said detection zone based on the image calibration indicia;
calculating a pixel density value of the test line of said detection zone, based on an intensity of said test line depicted in the at least one image;
obtaining an analyte concentration value corresponding to the pixel density value of the test line of said detection zone; and
communicating the obtained analyte concentration value to at least one of the patient or a medical service provider of the patient.

19. A method for screening a volume of biofluid of a patient for an at least one medical condition using the biofluid-based diagnostic screening system of claim 18, the method comprising the steps of:
implementing a user application residing in memory on an imaging device;
collecting a volume of the patient's biofluid using the biofluid collection device;
depositing an appropriate volume of the biofluid from the biofluid collection device onto the at least one sample pad of the test strip;
positioning the test strip on the strip placeholder within the test housing;
positioning the imaging device on the upper surface of the top panel of the test housing so that the image capture device is positioned over the imaging aperture of the top panel;
the user application capturing an image of the identification code of the test strip using the image capture device of the imaging device;
the user application determining the at least one test type to be performed on the volume of the patient's biofluid based on the captured image of the identification code;
the user application obtaining a local temperature relative to the imaging device;
the user application setting an appropriate calibration curve metric and a timer duration for each of the at least one test type, based on the at least one target analyte of the at least one test type as a function of the local temperature; and
for each of the at least one test type to be performed on the volume of the patient's biofluid, upon the associated timer duration being reached for said test type:
the user application capturing, via the image capture device of the imaging device, at least one image of the corresponding at least one detection zone of the test strip and the image calibration indicia;
the user application adjusting a white balance of the captured at least one image of said detection zone based on the image calibration indicia;
the user application calculating a pixel density value of the test line of said detection zone, based on an intensity of said test line depicted in the at least one image;
the user application obtaining an analyte concentration value corresponding to the pixel density value of the test line of said detection zone; and
the user application communicating the obtained analyte concentration value to at least one of the patient or a medical service provider of the patient.

20. The method of claim 19, wherein the step of the user application obtaining an analyte concentration value corresponding to the pixel density value of the test line of said detection zone, further comprises the step of the user application calculating the analyte concentration value by using the calibration curve metric as a function of the pixel density value.
